(19) United States
(12) Patent Application Publication
(10) Pub. No.: US 2007/0003930 A1
(43) Pub. Date: Jan. 4, 2007

(54) Handling of dna

(75) Inventors: Adam Long, Birmingham (GB); Peter Gill, Birmingham (GB); Tim Cox, Worcestershire (GB)

Correspondence Address: FITCH EVEN TABIN AND FLANNERY, 120 SOUTH LA SALLE STREET, SUITE 1600, CHICAGO, IL, 60603-3406, US

(73) Assignee: THE SECRETARY of STATE for the HOME DEPART- MENT (GB) and QINETIQ LIMITED (GB)

(21) Appl. No.: 10/545,752

(22) PCT Filed: Feb. 16, 2004
(86) PCT No.: PCT/GB04/00618

§ 371 Date: Jul. 27, 2006

(30) Foreign Application Priority Data
Feb. 15, 2003 (GB) 0303524.3

(51) Int. Cl. C12Q 1/68; C07H 21/04

(52) U.S. Cl. 435/6; 536/25.4

(57) ABSTRACT

A variety of methods are provided which use a silicon or silicon dioxide channel to extract DNA from a sample and then release it at a later point. The extraction channels are simple to manufacture and reliable in use. Prior art problems with entrainment of gas, liquid and solid material within channels are addressed. The techniques provide a convenient way of controlling the amount or concentration of DNA in the eluant.

This invention concerns improvements in and relating to the handling of DNA, and in particular, its capture by and release from surfaces. The surfaces may, more particularly be provided by microfabricated silicon channels.

There has been recent interest in the use of miniaturised components for performing the amplification stage of DNA analysis. In general the samples for amplification are prepared in other apparatus and then introduced into the device. Within chambers constructed in the device various processes are performed. The requirements for initial sample handling and processing outside of the device and the requirement for specifically designed chambers in the device represents a restriction on the range of applications to which such devices can be put and presents cost implications.

Some attempts have been made to extract DNA during its passage through a channel. Such techniques, however, use beads, projections, and other features within the channels to achieve the extraction; U.S. Pat. No. 6,440,725. Such techniques face problems in terms of their complexity, reliability in performance and consistency in performance between runs. Attempts have been made to extract DNA during its passage through a microfluidic chip. In particular, U.S. Pat. No. 6,440,725 describes a chamber in which there are filters, beads, glass wool, membranes, filter paper, polymers and gels. The DNA is extracted onto the surfaces of these structures. These structures will allow a plurality of fluidic paths between the input and outlet of the chamber. Firstly, in these structures it is difficult to avoid bubbles. Secondly, if a gas is flowed through the structure to separate batches of reagents, breakthrough often occurs along one fluidic path. The result is that pockets of liquid often remain in the chip when gas is flowed through the chip. This results in carry over of reagents between steps. Thus, for example, ethanol used in a wash step may be carried over into the eluent. It is well known that ethanol can inhibit subsequent PCR. In U.S. Pat. No. 6,440,725 the surface projections for trapping the DNA are introduced into the chamber either as part of the fabrication process or subsequently. In the present application describing an extraction channel, there are no such projections. The DNA is trapped on the walls of a smooth walled channel. For the case of a channel, the interaction of the sample with the trapping surface, i.e. the wall is well defined. This allows very reproducible sample preparation giving a well defined yield of DNA. This is important as the success of some PCR assays can be very sensitive to the amount of DNA present.

In addition, the flow of sample and reagent through a single channel is tolerant to bubbles within the sample. These are found to move smoothly through the structure.

The present invention considers and develops the possibilities for preparing the sample within a microfabricated device, instead of in other apparatus, using single flow path channels. In particular techniques and materials for DNA extraction, cleaning, isolation and extraction are provided. Amplification and subsequent analysis steps can then be performed. Success in achieving these aims gives rise to number of benefits and advantages. For instance, by fully integrating the preparation, amplification and potentially analysis of the results into such a device, a miniaturised system suitable for the analysis of forensic samples is provided. Such devices are beneficial in terms of their portability, ability to handle very small samples, ability to concentrate and handle very dilute samples and provide a variety of others benefits.

According to a first aspect of the invention we provide a method of extracting DNA from a sample, the method including:

providing an extraction channel;

Introducing the sample containing DNA to the extraction channel, passing the sample through the extraction channel and removing the sample from the extraction channel, at least a part of the DNA being retained by the channel and thereby being extracted from the sample; and

subjecting the extracted DNA to one or more further process steps;

wherein a single flow path for the sample is provided within the part of the extraction channel provided to retain DNA.

In this way the method is made less susceptible to problems with bubbles or solid material in the sample interrupting or altering the flow during extraction. A method which is more reliable in extracting the DNA and which is more consistent in its performance from one run to the next is provided as a result.

The surface area of the extraction channel may be predefined.

The extraction channel may have an inlet and an outlet, the distance along the channel between the inlet and the outlet being at least 10 times the shortest distance measureable between the inlet and the outlet.

The DNA may be accompanied in the sample by one or more impurities, such as PCR inhibitors. At least a part of the one or more impurities, such as PCR inhibitors, may remain in the sample and so passing through the channel, whilst the DNA is retained. The eluent may contain less of the one or more impurities, such as PCR inhibitors, than the sample.

The extraction channel may have a DNA retention capacity, the amount of DNA in the post-extraction eluate being less than or equal to the retention capacity of the extraction channel.

According to a second aspect of the invention we provide a method of extracting DNA from a sample, the method including:

providing an extraction channel;

introducing the sample containing DNA to the extraction channel, passing the sample through the extraction channel and removing the sample from the extraction channel, at least a part of the DNA being retained by the channel and thereby being extracted from the sample; and

subjecting the extracted DNA to one or more further process steps;

wherein the surface area of the extraction channel is predefined.

In this way the method provides for a known and consistent extent of DNA extraction from a sample and hence control over the amount of DNA in the eluent.

A single flow path for the sample may be provided within the part of the extraction channel provided to retain DNA.

The extraction channel may have an inlet and an outlet, the distance along the channel between the inlet and the outlet being at least 10 times the shortest distance measurable between the inlet and the outlet.

The DNA may be accompanied in the sample by one or more impurities, such as PCR inhibitors. At least a part of the one or more impurities, such as PCR inhibitors, may remain in the sample and so passing through the channel, whilst the DNA is retained. The eluent may contain less of the one or more impurities, such as PCR inhibitors, than the sample.

The extraction channel may have a DNA retention capacity, the amount of DNA in the post-extraction eluent being less than or equal to the retention capacity of the extraction channel.

According to a third aspect of the invention we provide a method of extracting DNA from a sample, the method including:

providing an extraction channel;

introducing the sample containing DNA to the extraction channel, passing the sample through the extraction channel and removing the sample from the extraction channel, at least a part of the DNA being retained by the channel and thereby being extracted from the sample; and

subjecting the extracted DNA to one or more further process steps;

wherein the extraction channel has an inlet and an outlet, the distance along the channel between the inlet and the outlet being at least 10 times the shortest distance measurable between the inlet and the outlet.

In this way the extraction channel is provided with sufficient length so as to achieve the desired amount of DNA extraction, whilst minimising the overall size of the extraction process.

A single flow path for the sample may be provided within the part of the extraction channel provided to retain DNA.

The surface area of the extraction channel may be predefined.

A single flow path for the sample may be provided within the part of the extraction channel provided to retain DNA.

The DNA may be accompanied in the sample by one or more impurities, such as PCR inhibitors. At least a part of the one or more impurities, such as PCR inhibitors, may remain in the sample and so passing through the channel, whilst the DNA is retained. The eluent may contain less of the one or more impurities, such as PCR inhibitors, than the sample.

The extraction channel may have a DNA retention capacity, the amount of DNA in the post-extraction eluent being less than or equal to the retention capacity of the extraction channel.

According to a fourth aspect of the invention we provide a method of extracting DNA from a sample, the DNA being accompanied in the sample by one or more impurities, such as PCR inhibitors, the method including:

providing an extraction channel;

introducing the sample containing DNA to the extraction channel, passing the sample through the extraction channel and removing the sample from the extraction channel, at least a part of the DNA being retained by the channel and thereby being extracted from the sample, at least a part of the one or more impurities, such as PCR inhibitors, remaining in the sample and so passing through the channel and/or irreversibly binding to the extraction channel; and

subjecting the extracted DNA to one or more further process steps to elute the extracted DNA into a post-extraction eluent, the eluent containing less of the one or more impurities, such as PCR inhibitors, than the sample.

A single flow path for the sample may be provided within the part of the extraction channel provided to retain DNA.

The surface area of the extraction channel may be predefined.

The extraction channel may have an inlet and an outlet, the distance along the channel between the inlet and the outlet being at least 10 times the shortest distance measurable between the inlet and the outlet.

The extraction channel may have a DNA retention capacity, the amount of DNA in the post-extraction eluent being less than or equal to the retention capacity of the extraction channel.

According to a fifth aspect of the invention we provide a method of extracting DNA from a sample, the method including:

providing an extraction channel, the extraction channel having a DNA retention capacity;

introducing the sample containing DNA to the extraction channel, passing the sample through the extraction channel and removing the sample from the extraction channel, at least a part of the DNA being retained by the channel and thereby being extracted from the sample; and

subjecting the extracted DNA to one or more further process steps to elute the extracted DNA into a post-extracion eluent, the post-extraction eluent containing DNA, the amount of DNA being less than or equal to the retention capacity of the extraction channel.

In his way the method provides a way in which the amount of DNA can be controlled to a desired level or amount, irrespective of the starting level or amount in the sample.

A single flow path for the sample may be provided within the part of the extraction channel provided to retain DNA.

The surface area of the extraction channel may be predefined.

The extraction channel may have an inlet and an outlet, the distance along the channel between the inlet and the outlet being at least 10 times the shortest distance measurable between the inlet and the outlet.

The DNA may be accompanied in the sample by one or more impurities, such as PCR inhibitors. At least a part of the one or more impurities, such as PCR inhibitors, may remain in the sample and so passing through the channel, whilst the DNA is retained. The eluent may contain less of the one or more impurities, such as PCR inhibitors, than the sample.

According to a sixth aspect of the invention we provide a method of extracting DNA from a sample, the method including:

providing an extraction channel;

introducing the sample containing DNA to the extraction channel, passing the sample through the extraction channel and removing the sample from the extraction channel, at least a part of the DNA being retained by the channel and thereby being extracted from the sample; and

subjecting the extracted DNA to one or more further process steps;

wherein the sample is provided in a liquid, the liquid having a viscosity of less than 10×10⁻³ kg/m/s.

In this way the sample is rendered suitable for passage through the extraction channel at acceptable flowrates.

A single flow path for the sample may be provided within the part of the extraction channel provided to retain DNA.

The surface area of the extraction channel may be predefined.

The extraction channel may have an inlet and an outlet, the distance along the channel between the inlet and the outlet being at least 10 times the shortest distance measurable between the inlet and the outlet.

The DNA may be accompanied in the sample by one or more impurities, such as PCR inhibitors. At least a part of the one or more impurities, such as PCR inhibitors, may remain in the sample and so passing through the channel, whilst the DNA is retained. The eluent may contain less of the one or more impurities, such as PCR inhibitors, than the sample.

The extraction channel may have a DNA retention capacity, the amount of DNA in the post-extraction eluent being less than or equal to the retention capacity of the extraction channel.

The one or more further process steps may elute the extracted DNA into a post-extraction elution, for instance, in a purified format at a concentration suited for further analysis.

The DNA may be at a first concentration in the sample and may be at a second concentration in a post-extraction elution. Preferably the concentration of DNA is higher in the post-extraction elution than in the sample.

In particular the first and/or second and/or third and/or fourth and/or fifth and/or sixth aspects of the invention may include any of the following features, options or possibilities.

The DNA may be extracted for forensic and/or medical and/or pharmacological and/or veterinary and/or bio-security consideration. The consideration may include the determination of at least a part of the sequence of the DNA. The sequences and/or base identities at one or more specific locations may be considered. The consideration may seek to link an individual to a sample or a sample to an individual. The consideration may seek to determine whether or not a person or animal has a particular medical condition or type of condition. The consideration may be to seek to identify a biological pathogen. The consideration may provide an indication of a positive or negative result. The consideration may provide an indication as to the likelihood of a condition applying. The consideration may give an indication as to the level or severity of a condition.

The sample may be collected from a site, particularly a site outside of an organism. The site may be a crime scene or a part there of. The location may be a surface or item. The sample may be collected from a person, particularly a blood sample.

The sample may be pre-prepared before introduction to the method, but preferably is introduced in a raw form. The sample may be introduced as blood, particularly blood introduced to the extraction channel.

The sample may have a volume of greater than 30 μL. The sample may have a volume of greater than 100 μL.

The extraction channel is preferably used to process the DNA in the sample and transport the DNA from one location to another.

The configuration of the extraction channel may be defined on the surface of the silicon wafer by a protective material, for instance a photoresist applied to the wafer. The extraction channel may be formed by etching, for instance, deep dry etching. The channel may then be coated with a layer of silicon dioxide, for instance 1 nm to 10 μm thick, preferably 50 nm to 1 μm thick. The extraction channel is preferably formed of silicon coated with a silicon dioxide layer. The extraction channel may be formed in a silicon wafer, particularly a p-type wafer, although n-type wafers can be used. The resistivity of the wafer may be between 0.0001 and 10,000 ohms.cm or more preferably between 1 and 10 ohms.cm. The silicon dioxide layer might be grown by exposure of the silicon to an oxidising ambient at elevated temperatures (e.g Oxygen gas at 1000° C.) A silicon dioxide film could also be deposited by chemical vapour deposition or by a plasma enhanced chemical vapour deposition. The silicon and/or silicon dioxide walls of the extraction channel may be provided with porous silicon in one or more cases. Preferably all such walls are so provided. The porous silicon may be provided on the whole or only part of a wall. The silicon wall may be provided with porous silicon prior to silicon dioxide growth or deposition. Porous silicon dioxide may be provided to increase the amount of DNA per unit area the extraction channel can retain. The porous silicon may be oxidised, at least in terms of its surface, to provide desired surface characteristics. An extraction channel through the full depth of the wafer may be formed. Preferably the wafer forms the side walls of the extraction channel. The wafer may form one of the base walls of the extraction channel. One or both base walls of the extraction channel may be formed by another component. The other component may be a glass plate and the wafer may be mounted on the glass plate. The other components could be a silicon wafer. In this way all the walls may be formed from silicon coated with silicon dioxide. A channel closed on both sides and at top and bottom is preferably formed The wafer and plate may be anodically bonded to one another. The plate may provide an inlet chamber for the extraction channel and/or an outlet chamber for the extraction channel.

Preferably the extraction channel consists only of the extraction channel walls. Preferably the walls are planar. Preferably the extraction channel is free of beads, projections or other such features. Preferably the single flow path prevents air bubbles remaining within the extraction channel, and ideally results in any air bubbles moving with the sample as it flows through the extraction channel. Preferably the single flow path prevents parts of a liquid remaining in the extraction channel after that liquid has been passed through the extraction channel. Preferably the single flow path prevents a part of a first liquid contacting a second liquid, particularly a second liquid which is passed through the extraction channel after the first liquid. Preferably the single flow path prevents solid material remaining within the extraction channel, and ideally results in any solid material moving with the sample through the extraction channel. Preferably the single flow path inhibits and ideally prevents blockages forming in the extraction channel.

The extraction channel may have a depth and/or side wall height of between 1 μm and 1000 μm. The depth and/or side wall height may, more preferably, be between 50 μm and 350 μm. The extraction channel may have a width and/or base wall extent of between 1 and 1000 μm, preferably between 10 and 500 μm, more preferably between 30 and 75 μm.

The extraction channel may have a length of between 1 mm and 10000 mm, preferably between 10 mm and 5000 mm, more preferably between 100 mm and 1000 mm. The extraction channel may have a surface area of between 0.1 and 150 cm². The surface area may be between 1 and 5 cm².

The extraction channel may have a volume of between 0.005 and 2500 mm³. The volume may be between 1 and 10 mm³.

The extraction channel may have an aspect ratio, depth and/or side wall height to width and/or base wall extent of between 1:1 and 20:1, preferably between 3:1 and 10:1 and ideally around 5:1.

The extraction channel may have a serpentine profile. The distance between the inlet and the outlet along the channel may be at least 10 times the shortest distance between the inlet and the outlet, more preferably at least 30 times.

Preferably the surface arc of the extraction channel is predefined so as to extract a predefined amount of DNA from the sample. Preferably the surface area of the extraction channel is predefined by knowing its surface area. Preferably the surface area is known by knowing the dimensions of the extraction channel. Preferably the surface area of the extraction channel is predefined as a result of the extraction channel design process. Preferably the surface area of the extraction channel is known as a result of the extraction channel not including or incorporating any features, as a part of itself or additional to itself, whose surface area is not known. Such surface areas may be not known where the dimensions, extent, number, profile or surface nature of the features are unknown.

The extraction channel may be pre-prepared before the sample is introduced. The pre-preparation may occur shortly before use and/or as part of the manufacturing process. The pre-preparation may involve contacting the extraction surface with an alkali, for instance NaOH. The alkali may have a concentration of at least 1 mM and more preferably of at least 5 mM. The pre-preparation may involve contacting the extraction channel with one or more liquids and/or one or more different volumes of the same liquid. The pre-preparation liquid or liquids may be moved through the extraction channel using a gas over pressure applied to the inlet. One or more volumes of water, preferably deionised, may be introduced to the extraction channel, preferably after an alkali. This may be so as to ensure efficient removal of the alkali from the channel.

The flow rate of the sample through the extraction channel may be controlled by the extraction channels cross-section. The flow rate of the sample through the extraction channel may be controlled by the pressure applied to the sample. Preferably both controls are used. The extraction channel cross-section may be consistent along its length or a restriction may be provided at one or more locations. Preferably any restriction any provides a single flow path.

The pre-preparation liquids and/or sample and/or eluent may be passed through the extraction channel by the application of pressure. The pressure may be an over pressure applied to the inlet to the extraction channel. The over pressure may be between 1 and 25 psi.

One or more volumes of water, preferably de-ionised, may be introduced to the extraction channel before the sample is introduced, The one or more volumes of water may be collected after passage through the extraction channel and may be used as a negative control in subsequent analysis and/or consideration of results.

The extraction channel may be subjected to a gas or airflow, preferably a flow of filtered high purity nitrogen. The gas or airflow may be applied between removal of one or more volumes of water and the introduction of the sample. The gas or airflow may be applied for between one and ten minutes.

The sample may provide the DNA in a mixture in the liquid phase including one or more chaotrophic salts. The mixture may further include detergent and water. The chaotrophic salt may be guanidine hydrochloride. The DNA may be provided in a sample having a high ionic strength. The sample may be provided in a liquid phase having a first pH, preferably a first pH which promotes retention of the DNA by the extraction channel. The sample may include one or more chemicals which disrupt protein structure. The sample may include one or more chemicals which disrupts protein structure and removes water molecules from the vicinity of the DNA molecules.

The sample may be provided in a mixture of a chaotrophic incorporating a mixture of one or more alcohols, such as ethanol and/or propanol. The sample may be provided in a mixture formed by mixing a Qiagen chemistry buffer with one or more alcohols, such as ethanol and/or propanol. Preferably the mixture is formed within the range of between one part alcohol to two parts Qiagen buffer and two parts alcohol to one part Qiagen buffer. More Preferably the mixture containing the chaotrophic salt is mixed with a further material, such as ethanol to reduce the viscosity of the sample.

Preferably the viscosity of the sample is between 1×10⁻³ and 10×10⁻³ kg/m/s.

Preferably the sample is introduced to the extraction channel via an inlet port. The inlet port may be provided by a tube or may be a reservoir, particularly in glass mount for the wafer in which the extraction channel is at least partially formed. A gas over pressure, for instance between 3 and 8 psi, may be applied to introduce the sample into the extraction channel and/or pass the sample through the extraction channel. Preferably the gas over pressure is used to move the sample into the extraction channel and is then released. Preferably the sample remains in the extraction channel for between ten seconds and twelve hundred seconds. Preferably the sample remains within the extraction channel for a time of between sixty and six hundred seconds. The extraction channel may be incubated whilst the sample is passing through the extraction channel. Incubation may occur at a temperature of between 10 and 80° C. and more particularly 70° C. plus or minus 3° C.

The sample may be introduced in a single volume. The sample may be introduced in multiple volumes.

The sample may have a volume of between 10 μL and 1000 μL. Preferably the sample size is in the range of 20 μL to 300 μL. The DNA concentration in the sample may be at least 0.001 pg per μL.

Preferably a gas over pressure is reapplied to remove the sample from the extraction channel. The sample may be removed from the extraction channel by flowing into an outlet port. The outlet port may be provided by a tube or may be provided by a reservoir, particularly a reservoir provided in the glass plate on which the wafer is mounted.

The steps of drying the extraction channel, introducing the sample to the extraction channel, allowing the sample to rest in the extraction channel and then removing the sample from the extraction channel may be repeated a plurality of times. The plurality of times may range between two and ten times.

The steps involving introducing the sample to the extraction channel, allowing the sample to rest in the extraction channel, introducing more sample into the extraction channel whilst simultaneously displacing/removing the first sample may be repeated a plurality of times. The plurality of times may be in the range between two and twenty times.

The extraction channel with DNA retained in it may be dried or otherwise cleared of unretained sample. PCR reagents may be introduced to the extraction channel to perform amplification of the DNA in the extraction channel. PCR may be started in the extraction channel and even taken to completion therein. The PCR reagents may themselves release the retained DNA from the extraction channel or may ne accompanied by further reagents for this purpose.

The extraction channel with DNA retained in it may be washed. The extraction channel may be washed by a buffered solution of high ionic strength. The extraction channel may be washed with a mixture of ethanol and chaotrophic salts. The extraction channel may be washed to remove proteins and/or cellular material and/or other impurities and/or inhibitors of PCR.

The channel may be washed using a Qiagen chemistry wash buffer. The volume of wash buffer of between 10 μL and 500 μL may be used. Preferably a volume of between 30 μL and 50 μL is used. The steps involving the introduction of a wash buffer, passing the wash solution through the channel, removing the wash buffer may be repeated a plurality of times. The plurality of times may be in the range of between two and twenty times.

Preferably the DNA is extracted from the sample by reversible binding with one or more parts of the extraction channel. The reversible binding may occur between the DNA and the silicon dioxide on the walls of the silicon extraction channel. Preferably the binding is made reversible by providing the DNA in a high ionic strength liquid, particularly a Qiagen chemistry buffer. Preferably the binding is made reversible by providing the DNA in a different pH to the pH at the time of the binding to the extraction channel. Preferably this second pH is different to the first pH used to promote retention of the DNA by the extraction channel.

The retained DNA may be eluted in a different liquid equivalent to the liquid of the sample. The retained DNA may be eluted with a buffer. The retained DNA may be eluted by a low ionic strength liquid, such as Tris HCL/EDTA and/or water The retained DNA may be eluted using a liquid at between 50° C. and 80° C. and more particularly 70° C. plus or minus 3° C. A single volume of liquid may be introduced to the extraction channel to elute the retained DNA. A plurality of volumes of eluent may be used. Between 1 and 10 eluent volumes may be used. The eluent may be introduced to the extraction channel through the same inlet as the sample was introduced through or may be introduced through a different inlet. The eluent may leave the channel through the same outlet as the sample or through a different outlet.

The eluent may flow through the extraction channel at a constant flow rate. The eluent may be allowed to rest in the extraction channel. The eluent may flow into the extraction channel so as to fill the extraction channel, be left for a period of time and then flow out of the extraction channel. The period of time may be between 10 seconds and 1200 seconds, but is preferably between 100 seconds and 800 seconds. The extraction channel may be incubated during the time the eluent is in the extraction channel. Incubation may occur as the eluent is introduced and/or removed and/or during any period the eluent is allowed to stand in the extraction channel.

The eluent may be introduced into the channel structure by applying pressure, particularly an over pressure. The over pressure may be released to allow the eluent to remain in the extraction channel. The eluent may be removed from the extraction channel by reapplying pressure, particularly an over pressure. The steps of introducing the eluent to the extraction channel, allowing the eluent to remain in the extraction channel and removing the eluent from the extraction channel may be repeated through a plurality of cycles. The plurality of cycles may be between two and twenty times.

Preferably the eluent is retained to form the post-extraction sample. This can then be subsequently processed either within and/or outside the device including the extraction channel.

The retained DNA may be eluted into a post-extraction sample whose volume is less than 100 μL. The post-extraction volume may be less than 50 μL. The post-extraction sample may particularly be less than 20 μL in volume.

The concentration of the DNA in the post extraction sample may be a factor of at least 5, more preferably at least 10 and potentially at least 20 increase on the concentration of DNA in the sample.

The post extraction eluent may contain a predetermined amount of DNA, for instance at least 2 ng of DNA from each 1 μL of blood in the sample.

Preferably the DNA in the post-extraction sample is not altered compared with the DNA in the sample. Preferably no adverse or detrimental effects occur as a result of extraction from the sample and/or retention by the extraction channel and/or release into the eluent. Preferably the integrity of the DNA is preserved from sample through to the post-extraction sample.

The impurities left in the sample may be dissolved species and/or suspended species and/or solid material. The impurities may be PCR inhibitors. The impurities may be haem and/or lead incorporating materials. The impurities may be debris, for instance debris associated with the cells from which the DNA does or does not originate and/or arising from the sample collection process. The impurities may be removed from the retained DNA by washing the extraction channel. The impurities may remain in the sample as it passes through the extraction channel and the DNA is retained by the extraction channel. Alternatively, the impurities may bind irreversibly to the silicon dioxide surface as the sample passes through the extraction channel.

One or more volumes of liquid may pass through the extraction channel separated from one another by a volume, for instance a slug, of gas. The gas may be air. The different volumes of liquid may be the same liquid or may be different liquids.

Preferably the retention capacity of the extraction channel is in part defined by its surface area. Preferably the extraction channel is formed to have a pre-determined retention capacity and/or retention capacity within a pre-determined range. The retention capacity and/or retention capacity range may be set so as to provide a particular maximum concentration of DNA in the post extraction sample.

An excess of DNA, compared with the retention capacity of the extraction channel, may be passed through the extraction channel. The concentration of DNA in the post-extraction sample may be at a pre-determined level.

The time taken for a sample to pass through the extraction channel may be used to control the level of DNA retained by the extraction sample.

The post extraction sample may be subjected to PCR. The PCR products may be subjected to electrophoretic based analysis. The PCR and/or electrophoretic based analysis may be performed outside the device incorporating the extraction channel, or more preferably, in one or both cases may be performed within the device incorporating the extraction channel.

The channel may be part of a system, for instance a system provided on an integrated chip. The system, for instance on an integrated chip, may provide one or more further functions. The further functions may include one or more of cleaning, washing, PCR, cell disruption or analysis.

Any and all references herein to DNA can be substituted by RNA; the invention being equally applicable thereto.

Various embodiments of the invention will now be described, by way of example only, and with reference to the accompanying drawings in which:

FIG. 1 is a method for fabrication of silicon glass chips

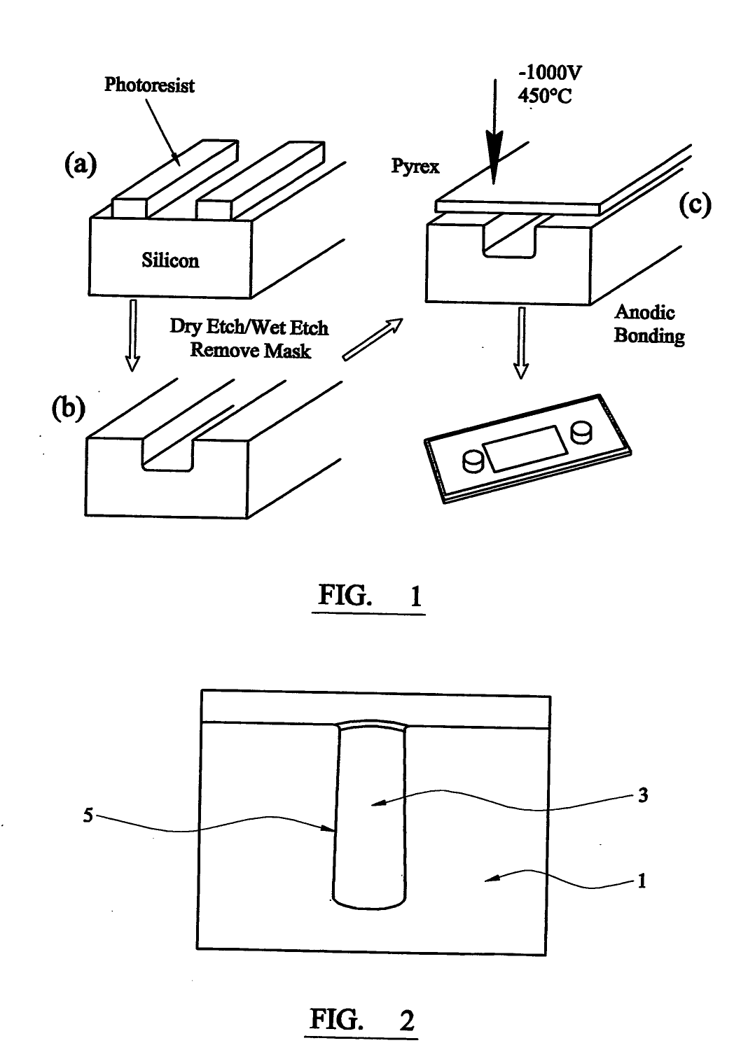

FIG. 2 is a cross section through a silicon wafer showing a deep dry etched channel;

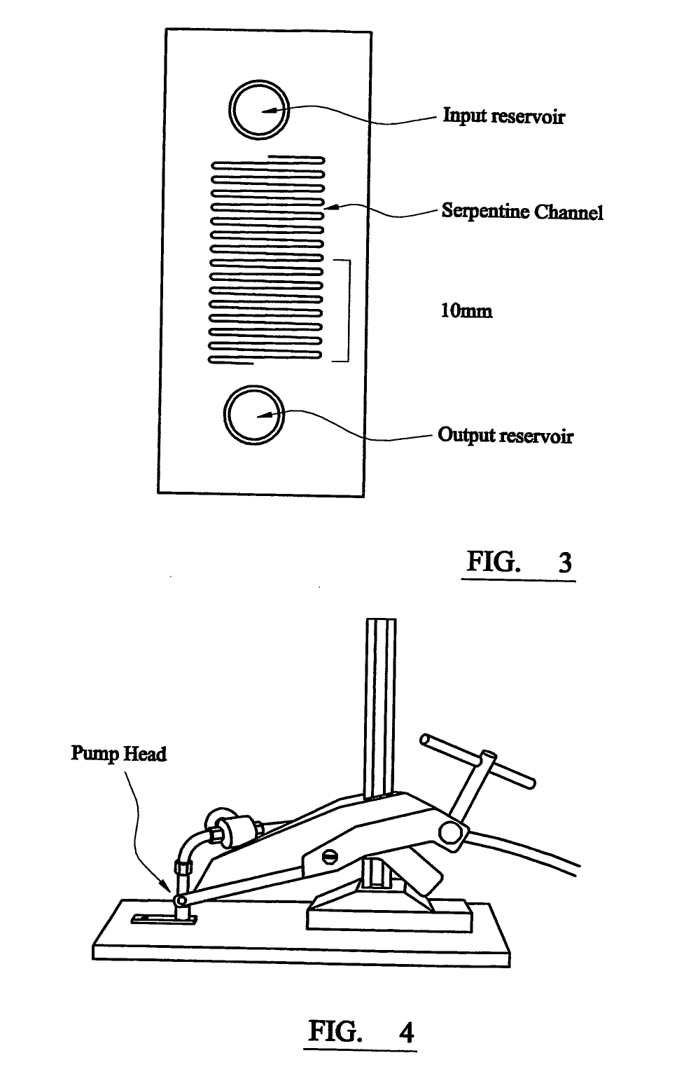

FIG. 3 shows in plan view a microfabricated silicon channel with a length of 300 mm;

FIG. 4 shows a picture of the pump head

FIG. 5 is an electropherogram illustrating the ability of extraction channels to retain DNA from certain sample forms;

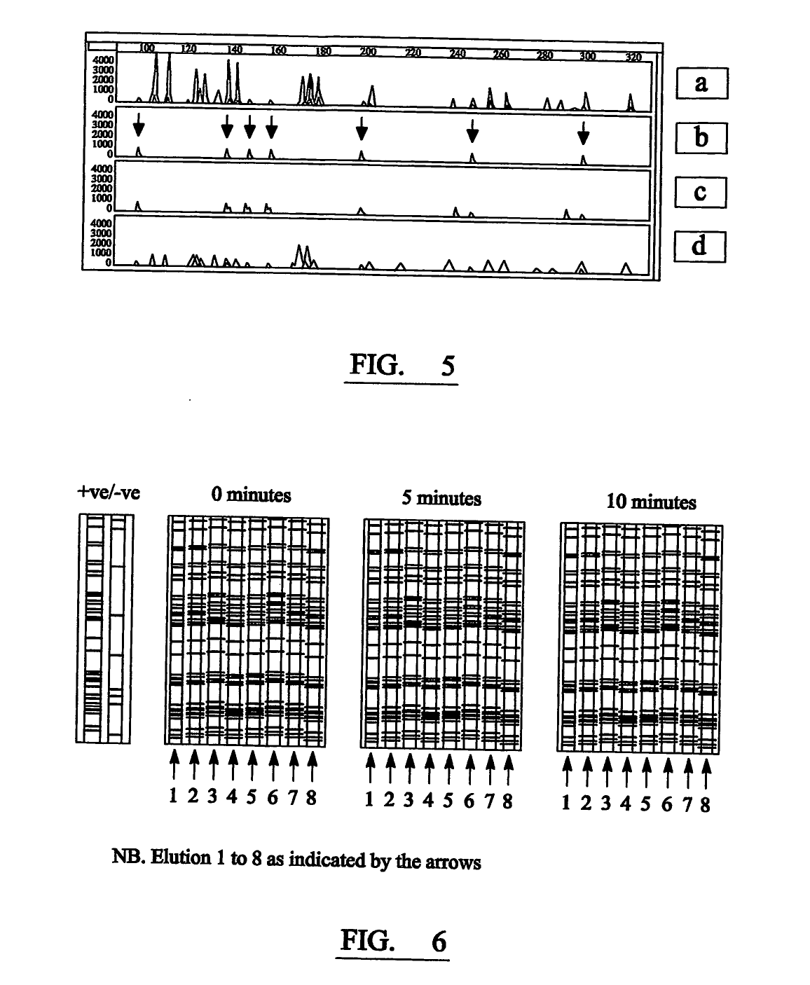

FIG. 6 illustrates the profiles generated for different incubation times of sample within the extraction channel;

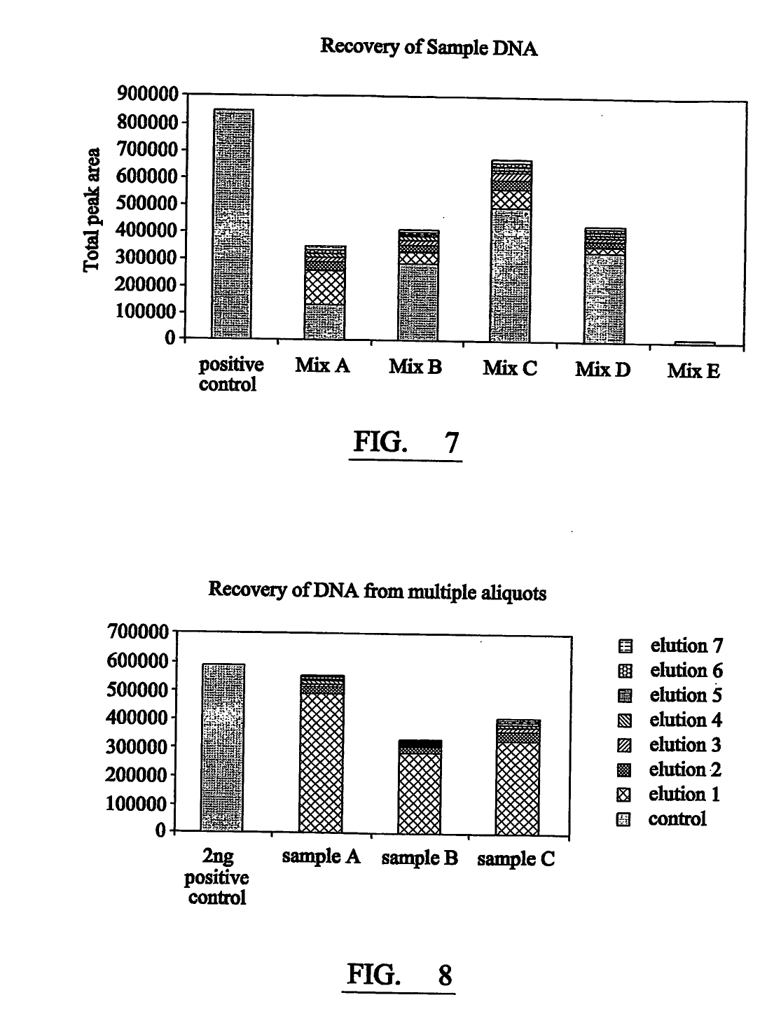

FIG. 7 is a graph of total peak area showing the extent of recovery with different elutions for samples including different amounts of ethanol;

FIG. 8 is a graph of total peak area illustrating recovery with different elutions for different initial DNA concentrations in the samples;

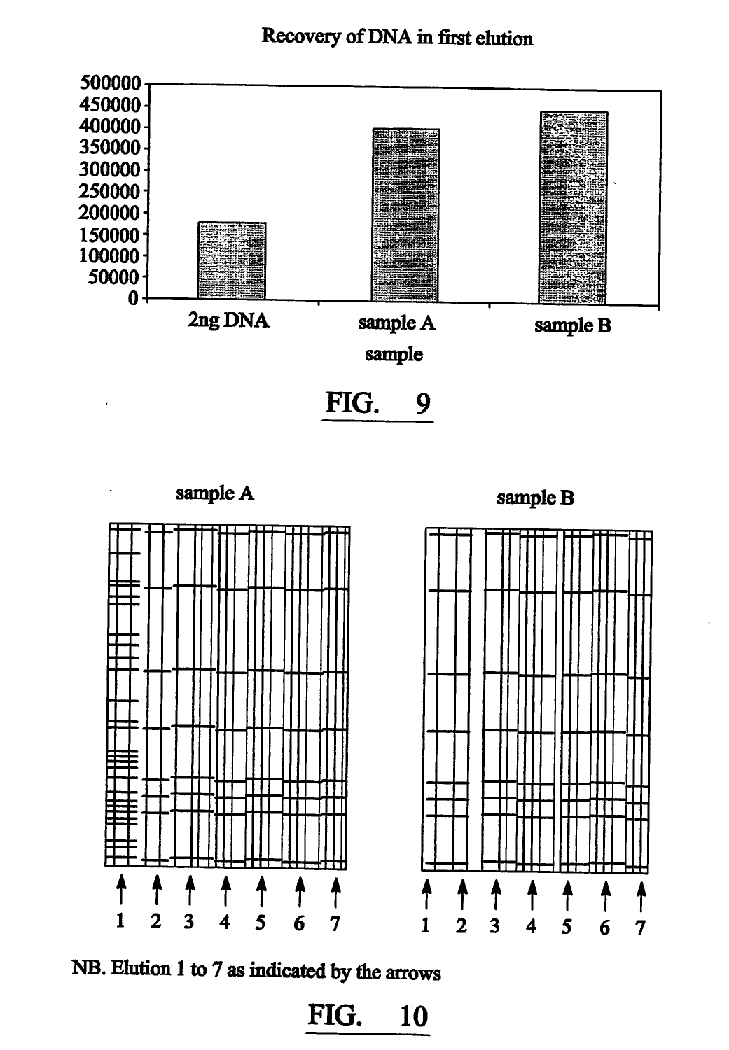

FIG. 9 illustrates the extent of recovery of DNA in a first elution from the extraction channel for whole blood samples;

FIG. 10 illustrates the gel profiles obtained for impure and purified samples; and

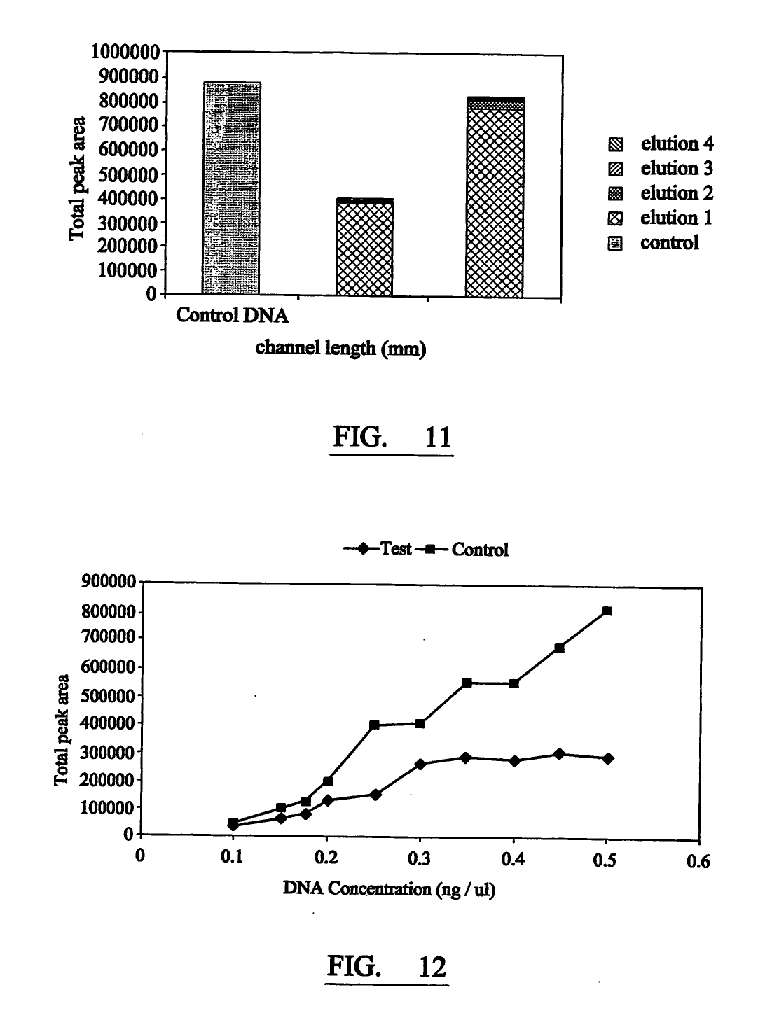

FIG. 11 illustrates the extent of recovery of DNA from two different extraction channels which are different in length as compared with the control.

FIG. 12 illustrates DNA binding saturation in 30 cm channels

In the context of forensic science, as with most scientific areas, there is an on going desire to reduce the costs involved in obtaining results and other useful information. Widely applicable apparatus having low manufacturing and/or operating costs is therefore desirable. There is also a need to provide faster analysis of samples, for instance, by the development of apparatus which can be used at a crime scene to speed up the overall collection, preparation and processing of samples. Highly portable apparatus for this function is therefore desirable too. Similar criteria apply in medical and/or pharmaceutical and/or veterinary and/or biosecurity contexts too.

Recently attempts have been made to miniaturise certain aspects of sample processing in the context of forensic science and other such contexts. This has involved the development of chips for PCR amplification. Work continues to develop such chips which are capable of collecting samples, cleaning samples and performing other tasks upon them. These highly complex and specialised features are often linked together and/or linked to inlets and outlets of such apparatus using channels in a device. The channels are merely used to convey the sample and/or other materials between one location and the next. No processing or other actions are performed on route and in particular no use is made of the channels other than to constrain the sample or other materials and so cause its transport. This is reflected in the A to B by the shortest possible approach route taken in such apparatus. Any interaction with the channel in the prior art was undesired, uncontrolled and avoided by all possible means. Any diminishing amount of material taken up by the channel walls would remain there asa the interaction is a one way process. Processing is carried out within chambers of the device and/or at the chips.

Within such devices considerable efforts have been made to keep the DNA apart from any silica surfaces present in a chamber, for instance. This is due to fears of such surfaces inhibiting PCR and/or damaging the DNA or reagents involved in the PCR process. Various polymer liners for such chambers have therefore been proposed.

Where techniques have been provided to extract DNA, they rely on beads, projections or other features provided within a channel and upon the surfaces of which the DNA is retained. The provision of such features provides a number of problems with such techniques. Firstly, there are increases in manufacturing complexity due to the need to provide these features within the channels. Secondly, the features create a significant number of separate flow paths that the liquid bearing the DNA may follow. The flow rate through any one of these flow paths and the small size of those flow paths render them susceptible to blocking by solid material in the sample and/or air bubbles. The nature of such blockages varies between runs and is not predictable. There are thus issues of consistency between runs and issues of reliability within any given run. The present invention stems from the realisation that the surface properties of single, simple profile channels can be harnessed to enable the channels themselves to perform a number of different processes useful in the context of sample collection and/or cleaning and/or release and that this can be achieved in a controlled and fully reproducible manner. Optimisation of channels for such uses, developments of such uses and various other improvements and possibilities are provided as a result of this work.

As a part of these developments, apparatus supporting reagents and methods have been developed which facilitate within a microstructure:
a) the capacity to trap/bind DNA without causing any adverse or detrimental effect with respect to DNA integrity;
b) the ability to enable washing solutions to be added and passed through the device so as to remove debris and inhibitors from the sample, ideally without compromising the amount of DNA retained within the structure;
c) the capacity to release bound DNA without disrupting DNA integrity into an elution stage.
d) the capacity to release a predetermined amount of DNA at an optimum concentration for subsequent analysis.

By achieving these possibilities the invention allows a variety of situations to be addressed which are not possible or are substantially impaired using prior art techniques.

In particular the invention renders it possible to concentrate initial samples containing DNA to levels more suited to subsequent processing. Frequently, DNA extraction methods involve sample volumes greater than 30 μL. This causes problems with existing systems as they possess a very limited ability to concentrate DNA solutions into smaller volumes. Silicon channels have the capacity to process samples within a much larger range and therefore have the following advantages. This allows samples which have been over diluted or for which the practicalities of recovery the sample resulted in a very low concentration of DNA to be successfully handled.

The invention also renders it possible to handle very small samples, or samples for which it is desirable to prepare only a small sample, as the sample volume requirements are low. Such situations include dried biological material which initially requires suspending in a liquid prior to extracting the small number of cells which provide the DNA. As the channels of the invention use small volumes, smaller suspensions can be made. This preserves the DNA concentration in samples where the number of cells is low.

The small nature of the samples which need to be handled in the various stages of the present invention also mean that reagent costs are reduced compared with the larger volume prior art techniques.

Furthermore, the manner of capture of the DNA means that effective removal of inhibitors from the solution which accompanies the DNA can be achieved. This is particularly important in forensic science applications and other low sample concentration situations as such inhibitors otherwise effect the efficiency of the amplification process and hence the standard of results obtained after PCR.

At the same time as providing these improvements the invention offers an efficiency gain by increasing the speed of processing and/or reducing the processing costs.

Microfabricated Channel Construction and Forms

Fabrication of Silicon/Glass Chips

Introduction

A wide variety of techniques and construct forms exist. The specific examples used in the development of the invention were fabricated as below.

Silicon glass chips were fabricated by the process shown schematically in FIG. 1. The desired pattern was defined in a thick layer of photoresist (a) applied to the top of the silicon The pattern transferred to the underlying silicon using a deep reactive ion etching process (b). Pyrex glass lids of thickness in the range 100 μm to 3 mm were then anodically bonded to the patterned silicon substrate (c). The processes are described in more detail below. FIG. 1 shows a method for the fabrication of silicon/glass chips

Patterning of the Silicon Substrate

The starting material was a 100 mm diameter p-type silicon wafer of orientation (100) and resistivity 1-10 ohm cm. The masking layer was defined in a photoresist layer (Hunts HPR-428) of thickness 7 microns using standard photolithographic techniques. The pattern defined here had a serpentine shape. The pattern was then transferred into the silicon using an STS deep dry etching machine. The etching process is a switched process in which a thin of polymer is first deposited and this is followed by a silicon etching step. The polymer protects the sidewalls from etching during the etching step. The repeated switching, approximately every ten seconds, allows deep features to be etched into silicon with high aspect ratio. The final step of the etching process, as used here, is a polymer deposition step. Thus the walls and bottom of the silicon channels will be coated with a very thin layer of a fluorocarbon polymer after this stage of the process. After the etching step, the photoresist mask is removed by 20 minutes treatment in an oxygen plasma followed by 10 minutes in fuming nitric add. A cross section through such a silicon wafer showing a high aspect ratio is shown in FIG. 2. It illustrates the silicon wafer 1, channel 3 and side walls 5 thereof. The deep channels are typically 125 μm deep×50 μm wide. FIG. 2 showing an extraction channel in cross section.

The deep channels obtained are typically 125 μm deep and 50 μm wide.

A layer of silicon dioxide of thickness 100 nm is grown on the exposed surface of the silicon by placing the silicon into a furnace containing oxygen at a temperature of 1000° C.

Anodic Bonding of Glass Lids to the Silicon Substrates

The silicon is cleaved up into single chips. These were then anodically bonded to glass plates of Coming 7740 glass of thickness in the range 0.1 to 3 mm with access holes drilled through the glass to form input and output ports to allow access to the two ends of the channels in the silicon chips.

In one embodiment the glass plate contains two 5 mm diameter drilled holes which permit access/egress to the ends of the channel. These act as inlet/outlet reservoirs during operation and have a capacity of about 75 mm³.

In a second embodiment, the glass is of thickness 1 mm and the inlet/outlet holes are on order 0.5 to 1 mm in diameter. Plastic tubes are then glued into these holes to form. Samples can then be flowed through the structure in a continuous mode, e.g. by connecting the inlet tube to a syringe controlled by a syringe driver.

Prior to anodic bonding the glass was cleaned by ultrasonicating in isopropyl alcohol for ten minutes. The glass and silicon chips were then cleaned in a 2:1 mixture, by volume, of 98% sulphuric acid and 35% hydrogen peroxide at 80° C. for ten minutes. The glass and silicon were rinsed in copious amounts of de-ionised water and blown dry in a stream of filtered nitrogen. All chemicals used were electronic grade. Bonding was performed in ambient air in a clean air cabinet at 430° C. and at an applied voltage of 700 volts. Electrical contact was made to the glass via a portion of silicon wafer with the rough back surface of the wafer next to the glass. In this way, a distributed multipoint contact was achieved. Typically, the bonding current was 600 μA falling to 100 μA for a chip of area 10 cm² over the ten minute period that the bias was applied.

A wide variety of channel lengths can be fabricated in this way and a variety of channel patterns are also possible. By way of example, channel lengths of 25 mm, 300 mm and 1000 mm have been fabricated in this way. An example featuring a 300 mm long channel is shown in FIG. 3. It features an input reservoir 7 which is linked via the serpentine channel 9 to the output reservoir 11

A typical microfabricated silicon channel structure as shown in FIG. 3 occupies a small area due to its small dimensions. Despite this however, the surface area can be relatively large. For a 1000 mm channel with a similar serpentine structure the surface area can be calculated as around 3 cm². A 1000 mm channel can hold approximately 6.25 μL of solution. As small volumes such as these can be difficult to introduce and convey through the channel an air pressure pump has been designed for this purpose. The sample is first pipetted into the input reservoir which can hold a maximum of about 65 microlitres (mm³) of sample. The sample is then driven through the channel by applying a positive pressure to the space above the sample in the input reservoir. The pump head is fitted with an o-ring washer which when lowered into position over the inlet reservoir creates an air tight seal. A positive gas over-pressure is then applied via a tube connected to a pressure regulated supply of filtered nitrogen. The pressure actively pushes solution from the inlet reservoir through the channel to the outlet reservoir. The solution may be removed from the outlet reservoir for further analysis. FIG. 4 shows a picture of the pump head.

Basic Reagent and Improved Reagent Set Ups

The general principle of the reagent system used is that of the Qiagen extraction chemistry where large scale columns are used; at least one order of magnitude greater in dimension than the present situation. The chemistry is well documented in Kelly, M. R., 1995. Rapid genomic DNA purification from Drosophila melanogaster for restriction and PCR. Qiagen News; 1, p 8-9. The applicant has made a number of improvements over this, however, to address problems in the use of this technology in the context of microfabricated channels.

Essentially the process involves suspending a DNA sample in a mixture of chaotrophic salt, such as guanidine hydrochloride, detergent and water. Chaotrophic salts disrupt protein structure and remove water molecules from the vicinity of the DNA molecules. This creates an environment that is high in ionic strength. As such it can be used to encourage DNA molecules to bind to a silica (silicon dioxide) matrix.

To address issues of viscosity in the context of microfabricated channels, whose dimensions are an order of magnitude smaller than the prior art contexts of use, a mixture of ethanol and chaotrophic salt containing a far higher level of ethanol has been developed. This mixture has a lower viscosity that pure chaotrophic salt alone which leads to an increase in flow rate through the structure resulting in an increase in the speed of action. This may increase the speed of extraction.

Further details of the extraction chemistry are provided below in the methodology and examples.

Methodology

(i) Obtaining Sample—Channel Based Route

The following procedure was used to extract the DNA from the sample using the channel based route of the present invention. A serpentine silicon channel supplied of the type described above was used.

Channel Pre-Wash

1. 2×20 μl aliquots 10 mM NaOH were introduced into the inlet port and passed through the channel using 12 psi gas over pressure.
2. 1×20 μl 18.2MΩ water and then 4×30 μl 18.2MΩ water were added and eluted as in step 1. All elution samples from stages 1+2 were discarded.
   Extraction Procedure
3. 20 μL 18.2MΩ water was passed through the channel using 12 psi. The evacuated sample was collected from the outlet port and stored for later analysis as a negative control.
4. The channel was exposed to a continuous airflow from the pump for 4 minutes.
5. ‘D’ μL of sample DNA (DNA diluted in a mixture of Chaotrophic salt+ethanol) was added to the inlet port.
6. A 5 psi gas overpressure was applied until the sample filled the channel. The air pressure was then released.
7. The sample was incubated inside the channel for ‘Y’ minutes.
8. A gas overpressure was reapplied to the inlet port and the sample was evacuated. The sample was discarded.
9. Repeat steps 5-8 were carried out ‘N’ times in total.
10. 30 μL Qiagen wash buffer was flowed through the channel. The waste was discarded.
   Elution Procedure
11. 20 μL 1× TrisHCl/EDTA (elution buffer) @ 70° C. or 20 μL 18.2MΩ water @ 70° C. was introduced into the channel using 12 psi.
12. The channel structure was placed into a humid hybridisation cabinet pre-set to 70° C. and left to incubate for 1 minute before being removed.
13. A 12 psi overpressure was reapplied to the inlet port until the sample was fully evacuated. This was collected during evacuation, and stored for later analysis.
Repeat steps 11 to 13 ‘Q’ times.
   (ii) Obtaining Sample—Qiagen Route Using Qiagen Extraction Columns

The following method was employed to extract DNA from liquid blood. It is generally referred to as the Qiagen extraction method and uses QIAmp spin columns. The method was used to provide samples for comparison purposes.
1. 1× TrisHCl/EDTA was incubated in a water-bath at 70° C.
2. 1 μl liquid blood was placed into a 1.5 mL tube.
3. 32 μl PBS, 4 μl Proteinase K and 32 μl AL buffer were then added.
4. The sample was mixed thoroughly by vortexing for 15 seconds.
5. The sample was then incubated in a water-bath at 70° C. for 10 minutes.
6. After removal from the water-bath excess moisture was removed using a tissue.
7. The sample was briefly pulse-spun to bring liquid down from the lid.
8. 32 μl ethanol (96-100%) was added and mixed by vortexing.
9. The sample was then spun down again.
10. The entire liquid content was transferred to an empty QIAamp spin column.
11. The sample was centrifuged for 1 minute at 6000 g.
12. The spin filter basket was transferred to a clean collection tube. (The used collection tube was discarded).
13. 80 μl AW1 was added to the spin column.
14. The sample was centrifuged for 1 minute at 6000 g.
15. The spin filter basket was transferred to a clean collection tube. (The used collection tube was discarded).
16. 80 μl AW2 was added to the spin column.
17. The sample was centrifuged for 3 minutes at 13000 g.
18. The spin filter basket was transferred to a dean eppendorf. (The used collection tube was discarded).
19. 20 μl of 1× ABD TE at 70° C. was added to the spin column.
20. The sample was incubated in a 70° C. water-bath for 5 minutes.
21. The sample was centrifuged for 1 minute at 6000 g.
22. The spin filter basket was discarded.
23. The eppendorf was capped and stored in a fridge.
   (iii) Eluted Sample Amplification

This method is used to amplify DNA present in eluted samples. The amplification protocol is given in the SGMplus amplification kits supplied by Perkin Elmer.
1. Samples extracted via the Qiagen method or chip method were made up to 20 μl using SDW.
2. Positive controls were made containing the same amount of DNA as those that were used in the individual experiments. Negative controls contained 20 μl SDW alone.
3. All samples were made up to 50 μL by adding 30 μl SGMplus multimix from the SGMplus kit. (The multimix contained all the PCR ingredients except for the DNA sample.

4. The mixtures were then amplified using a thermocycler programmed as described:

| 95° C. for 11 minutes - to activate the Taq Gold. | | |
|---|---|---|
| 94° C. for 1 minute (denaturation) | | |
| 59° C. for 1 minute (annealing) | {close oversize brace} | for 28 cycles |
| 72° C. for 1 minute (extension) | | |
| 60° C. for 45 minutes | | |

4° C. Hold - To keep the products cool until they are analysed.

Polyacrylamide Gel Electrophoresis

This method is used to separate PCR products following amplification. Each sample generates a profile corresponding to a series of alleles. Each allele generates a peak area. The combined total peak area for a sample profile is directly proportional to the amount of DNA present and therefore acts as a means of DNA quantification.

Once samples have been extracted by either experimental methods (i) or (ii), they are PCR amplified as described in experimental method (iii). All samples are made up to 20 μl using SDW and added to 30 μl SGM plus multimix before undergoing PCR. (Total reaction volume=50 μL). A control sample is also amplified using the same multimix and cycling conditions. Samples, which undergo PCR amplification, can be quantified using total peak areas.

To determine the amount of DNA present in each sample, the PCR products were run on a 377 gel to produce gel profiles. These were analysed using low analysis parameters (low threshold) so that very small peaks corresponding to the specific alleles of interest could be detected. The peak areas for all SGMplus loci were added together for each sample to give a total peak area. The total peak area for each elution was compared to that from a control sample to determine what proportion of DNA had been eluted.

### EXAMPLES AND DEMONSTRATIONS OF IMPROVEMENTS

### Example 1

### Illustration of Role of Extraction Chemistry in Systems Function

In this example a comparison of silicon channel extraction performance in relation to DNA diluted in (a) SDW and (b) Qiagen AL buffer was made. As such, two mixtures of DNA were made:

(1) 0.1 ng/μL DNA in 18.2 MΩ SDW

(2) 0.1 ng/μL DNA Qiagen AL buffer

15 μL aliquots of each sample were kept as control samples and were not processed through the extraction channel. These were called samples A and C respectively.

For the experiment, a 15 μL aliquot of mixture 1 was taken through the extraction protocol using the following conditions:

| | | |
|---|---|---|
| Volume of sample (μL) | D | 15 |
| Number of sample volumes added | N | 1 |
| Incubation time of sample (m) | Y | 2 |
| Number of elutions | Q | 4 |

The experiment was repeated for mixture 2 and so resulted in 4 elutions for each of the two experiments.

Following extraction, PCR was carried out on elution 1 from each experiment together with the two control samples A and B (2 ng Control DNA in 20 μL SDW), as detailed above. All PCR products were separated by gel electrophoresis, as detailed above, producing gel profiles which were subsequently analysed to produce electropherograms. PCR perfomred in this way, amplifies 11 separate regions (loci), resulting in up to 22 separate peaks for a heterozygotic sample (See profile A FIG. 5). X axis is a measure of the allele length (base pairs), Y axis is a measure of peak height. To separate out the alleles and to confirm allele identity, a sizing control ladder containing DNA fragments of known sizes are added to each sample prior to running (as indicated by the 7 arrows in profile b). The presence of large peaks corresponding to alleles indicate a successful amplification. Where allele peak heights are smaller, this is indicative of less DNA being present in the initial starting sample. The results for this example are illustrated in FIG. 5.

Electropherogram (a) shows the control profile for DNA in SDW. This represents the total amount of DNA present in the sample prior to processing.

Electropherogram (b) shows the profile obtained from an elution derived from the initial addition of DNA diluted in SDW. The lack of a profile suggests that no DNA was present in the elution. This indicates that the channel did not bind DNA from this particular sample. Only the internal sizing peaks as indicated by the arrows are present.

Electropherogram (c) shows the control profile for DNA in Qiagen buffer (not passed through the extraction channel. The buffer contains ethanol, which is known to inhibit PCR and therefore no profile is seen. (once again, only the control peaks are present.

Electropherogram (d) shows the profile obtained from an elution derived from the initial addition of DNA in Qiagen buffer. The presence of a profile exemplified by the presence of peaks corresponding to allels, suggests that DNA was present in the elution, and therefore must have been trapped within the channel during the extraction phase. The presence of PCR product also demonstrates that ethanol from the wash has been removed as this would otherwise cause PCR inhibition.

Although the size of the peaks and therefore total peak area within electropherogram (d) (total peak area 195093) are smaller than those in the control sample electropherogram (a) (total peak area 556951) the Qiagen buffer clearly encourages DNA to bind to the channel surface. Furthermore, the bound DNA is subsequently released when the elution buffer is added. SDW does not contain chaotrophic salt and therefore the conditions required for DNA trapping are not met. This results in no DNA being trapped and presumably none or very little being present in the elution.

This example demonstrates that DNA can be trapped within a silicon channel structure whose walls are coated with silicon dioxide using the Qiagen chemistry, that the DNA can be recovered from the channel by adding a low ionic strength elution buffer and that the use of such a buffer does not interfere with DNA integrity, as it is possible to amplify eluted DNA via the PCR reaction.

Further optimisation of the technique is now demonstrated.

### Example 2

### Illustrating the Effect of Increasing the Incubation Time on the Amount of DNA Recovered as Compared with the Control

In this example, DNA samples have been incubated inside the channel for (a) 0 minutes(simply addition of the sample followed by immediate removal), (b) 5 minutes and (c) 10 minutes. In total, eight elution washes were carried out for each incubation time. Each elution underwent PCR and then gel images were produced following separation on a flat bed gel electrophoretic sequencer. The results are illustrated in FIG. 6 with +ve,−ve controls, 0 min, 5 min and 10 min incubations (with 8 numbered elutions for each). The results show that as incubation time was increased more DNA was present in the initial elution (higher intensity of signal corresponding to lane 1) and in later elutions lane 2-4, (higher intensity and more elutions showing a profile), thus supporting more DNA as having been taken up by the channel. The signals in the 10 minute incubation run are stronger across a number of elutions when compared with the 0 minute incubation in particular.

To establish the total take up compared with the control sample, an electropherogram was created for the positive control and a total peak area was calculated. (1,040,444—total peak area). This represents the total amount of DNA in the control sample and is equal to the total amount of DNA added prior to each extraction.

Electropherograms were constructed in a similar fashion for each set of the eight elutions.

The peak area for the first elution was compared to the control value to highlight the difference in DNA concentration between each incubation time, with the results reported in Table 1. A dear illustration of improved take up and release into the first elution is demonstrated with increased incubation time.

TABLE 1
Peak areas and % recovery for elution 1for each incubation time.
Incubation time (minutes)

| 0 | | 5 | | 10 | |
|---|---|---|---|---|---|
| Peak | % | Peak | % | Peak | % |
| Area | recovery | Area | recovery | Area | recovery |
| 279970 | 27% | 497597 | 48% | 576602 | 55% |

The sum of the peak areas for each of the first eight elutions was also established to compare the total DNA recovery extent with time. The results are presented in Table 2. A clear indication as to increased total take up and release with increased incubation time is provided. No detrimental effect on the chemistry or DNA obtained was detected with the increased incubation time.

TABLE 2
Total peak areas and % recovery for elution
1-8 for each incubation time.
Incubation time (minutes)

| 0 | | 5 | | 10 | |
|---|---|---|---|---|---|
| Peak | % | Peak | % | Peak | % |
| Area | recovery | Area | recovery | Area | recovery |
| 373543 | 36% | 738778 | 71% | 979585 | 94% |

### Example 3

### Illustrating the Benefits of an Improved Buffer

As well as considering use of the established Qiagen buffer, improved alternatives were sought. Included in these were the consideration of DNA samples which were diluted in mixtures of Qiagen buffer according to prior art specifications and additional ethanol. Examples of the buffer make-ups considered are illustrated in Table 3.

TABLE 3
Shows the mixture ratios of Qiagen buffer:Ettianol.

| Mix | Ethanol (μL) | Qiagen buffer (μL) | Ratio |
|---|---|---|---|
| A | 0 | 18 | 0:1 |
| B | 6 | 12 | 1:2 |
| C | 9 | 9 | 1:1 |
| D | 12 | 6 | 2:1 |
| E | 18 | 0 | 1:0 |

The samples were processed through the silicon channel according to the extraction protocol. All elutions underwent PCR and gel separation. The combined peak areas for each experiment from the resulting electropherograms were compared to the total peak area from a positive control DNA. (820,000—total peak area). The results are shown in table 4.

TABLE 4
Total peak areas and % recovery for elution 1-8 for each experiment mixture.
Sample dilution mixture

| A | | B | | C | | D | | E | |
|---|---|---|---|---|---|---|---|---|---|
| Peak | % | Peak | % | Peak | % | Peak | % | Peak | % |
| Area | recovery | Area | recovery | Area | recovery | Area | recovery | Area | recovery |
| 337217 | 41% | 429680 | 52% | 732260 | 89% | 532180 | 65% | 1640 | 0.2% |

The peak areas for each elution in turn are plotted on the bar graph of FIG. 7 in relation to each of the experimental mixtures. The results show that adjusting the mixture to a 50% ratio of Qiagen buffer and Ethanol enhances the amount of DNA which is trapped in the channel. This is highlighted by the fact that the mixture combination gave the highest % recovery of DNA following elution from the channel. Benefits in terms of the ease with which the solution could pass through microfabricated devices, reducing the time taken to process each sample, were also observed for the high ethanol content mixtures.

### Example 4

### Illustrating the Use of Microfabricated Channels to Concentrate DNA from Multiple Samples

In this illustration DNA samples were prepared in a mixture of 50% Qiagen buffer and Ethanol at the concentrations set out in Table 5.

TABLE 5
DNA concentrations of sample solutions.

| Sample | DNA amount | DNA Concentration | |
|---|---|---|---|
| A | 2 ng in 200 μL | 0.01 | ng/μL |
| B | 1 ng in 200 μL | 0.005 | ng/μL |
| C | 2 ng in 20 μL | 0.1 | ng/μL |

A control sample, consisting of 20 μl of 0.1 ng/μl Control DNA in SDW, was amplified and analysed.

For each of the test DNA samples, 20 μL aliquots were sequentially incubated in the channel until the entire volume had been added. (Variable ‘N’, experimental method, i=10 pluralities for a total sample input volume of 200 μL). The DNA samples were then removed in a series of 7 elutions before undergoing PCR and separation.

The peak areas for each elution, for each sample dilution were calculated and compared to the total peak area derived from a 2 ng control DNA, (578,115—total peak area). This data is shown in Table 6. NB. The % recovery for solution B is calculated from the 2 ng control DNA however solution B only had 1 ng of DNA present. The total peak area derived from a given amount of DNA is directly proportional, therefore to reflect the difference in DNA amount between the control and dilution B, the total peak area for the control was divided by 2. The % recovery data presented for dilution B is normalised.

TABLE 6
Shows the total peak area and % recovery of DNA for each elution, for each
sample dilution compared to the total peak area for a 2 ng control.

| | DNA dilution | | | | | |
|---|---|---|---|---|---|---|
| | A | | B | | C | |
| | | % | | Normalised % | | % |
| | Peak Area | Recovery | Peak Area | Recovery | Peak Area | Recovery |
| Elution 1 | 491631 | 85.04% | 280154 | 96.92% | 329320 | 56.96% |
| Elution 2 | 24290 | 4.20% | 20204 | 7.0% | 30669 | 5.30% |
| Elution 3 | 11340 | 1.96% | 5135 | 7.18% | 14325 | 2.48% |
| Elution 4 | 4927 | 0.85% | 4517 | 1.56% | 4890 | 0.85% |
| Elution 5 | 2865 | 0.50% | 5764 | 2.00% | 3170 | 0.55% |
| Elution 6 | 3942 | 0.68% | 4654 | 0.16% | 4692 | 0.81% |
| Elution 7 | 3056 | 0.53% | 967 | 0.34% | 5094 | 0.88% |
| Total | 542051 | 94% | 321395 | 96% | 392160 | 68% |
| Peak area | | | | | | |

Once again, the peak areas for each individual elution, for each sample dilution are plotted in a bar graph as shown in FIG. 8.

This means that the total % recovery and total amount of DNA (g) recovered from each dilution, for elution 1 only was as follows:

| | | |
|---|---|---|
| Dilution A | 85.04% | ≈1.70 g DNA from 2 ng total DNA |
| Dilution B | 96.92% | ≈0.96 g DNA from 1 ng total DNA |
| Dilution C | 56.96% | ≈1.13 g DNA from 2 ng total DNA |

This result demonstrates the that recovery of DNA is possible even when the DNA concentration is very low (≈0.005 ng/μL). Potentially greater recovery occurs for the same amount of DNA in a large sample compared with a small sample, and the extraction method reliably concentrates DNA dilutions. This is evident by the concentration of sample B which demonstrates a 10 fold increase in concentration following elution in 20 μL.

### Example 5

### Illustrating Direct Extraction of DNA from Whole Blood

It is desirable for the system to function on biological samples directly, as well as on samples previously extracted from the original biological samples. This would allow the system to work on liquid whole blood, for instance.

1 μL of whole liquid blood was taken in duplicate and processed according to the experimental method (ii) for extraction using Qiagen up to and including instruction 9. This produces a crude extract containing Cell debris, haem, PBS, Proteinase K and DNA. The sample was then taken through experimental method (i) for channel based extraction. A total of eight elutions were performed for each sample and these underwent PCR and gel separation.

The peak areas for each elution, for each sample were calculated and compared to the total peak area derived from a 2 ng control DNA sample, (170,000—total peak area). This data is shown in table 7.

TABLE 7
Total peak area and percentage recovery of
DNA as compared to 2 ng positive control.

| | First elution | | | |
|---|---|---|---|---|
| | | % Control | Total elutions | |
| Sample | Peak area | DNA | Peak Area | % Control DNA |
| A | 397264 | 233.68% | 688821 | 405.18% |
| B | 443686 | 261.00% | 598315 | 351.97% |
| Control | — | — | 170,000 | 100% |

The peak areas for the first elution are displayed in FIG. 9 together with the positive control for comparison.

Samples A and B show respective peak areas from elution 1 equivalent to 397,264 and 443,686. Comparing these values with the total peak area from the 2 ng control suggests that samples A and B contain ≈2.5 times the amount of DNA. This equates to approximately 5 ng DNA in the first elution.

This result demonstrates that DNA can be routinely extracted from liquid whole blood using the silicon channel in combination with Qiagen chemistry and the amount of DNA extracted issuitable for immediate PCR amplification. It also implies that in the silicon extraction method inhibitors can be removed from samples during the extraction and washing phases. Haem present in red blood cells, for instance, is a powerful inhibitor of PCR. Following extraction, the elutions have successfully amplified demonstrating a lack of inhibition. This suggests that no or very little haem is present in the elutions following extraction.

### Example 6

### Illustrating the Extraction of DNA from Samples Known to be Contaminated with a PCR Inhibitor

The ability to remove contaminants and potential inhibitors of PCR is a desirable feature for any DNA analysis process. In this example, DNA samples containing a known inhibitor of PCR is extracted and purified using a silicon channel. This reflects the real world problem that samples collected from crime scenes are often contaminated with substances that inhibit PCR for example heavy metals, such as lead.

Bearing this in mind, initial experiments were carried out to determine what concentration of lead nitrate inhibited PCR. Amplifications were carried out with 2 ng control DNA adulterated with increasing amounts of lead nitrate. This investigation showed that DNA samples containing less than 5 ng/μL lead nitrate successfully amplified during PCR. Samples that contained above 12 ng/μL lead nitrate showed complete PCR inhibition.

As a result of these initial findings, a 2 ng DNA sample was adulterated with lead nitrate equivalent to 40 ng/μL (sample B) A duplicate 2 ng control DNA sample was also prepared but was not adulterated with lead nitrate.(sample A)

The samples were taken through the experimental method for channel based extraction. A total of eight elutions were performed for each sample and these underwent PCR and gel separation. The respective profiles for each elution (no. 1-7 in FIG. 10), for samples A and B are shown in FIG. 10. The result for sample A shows the profile obtained for the control i.e. when no lead nitrate was present in the DNA sample and therefore no PCR inhibition is seen. Lane 1 contains a strong profile. Some DNA is also seen in lanes 2 and 3 corresponding to elutions 2 and 3. Sample B shows the presence of a weak DNA profile in lane 1 (elution 1), indicating partial amplification. The original DNA sample contained 40 ng/μL lead nitrate, enough to completely inhibit PCR. The presence of a DNA profile however suggests that the amount of lead nitrate has been reduced to below 12 ng/μL but greater than 5 ng/μl following extraction. This means that between 28-35 ng/μl (1400-1750 ng total) lead nitrate has been successfully purified from the original sample equating to approximately 70% removal.

### Example 7

### Illustrating the Variation in Extracion with Different Length Channels

Each silicon channel has a fixed length and therefore has a fixed surface area. The surface area of the channel should determine how much DNA can be trapped during the sample incubation phase and therefore channel length should predetermine the total possible amount of DNA that can be extracted.

To investigate this issue three identical DNA samples were made up. Each contained a 2 μL aliquot of control DNA diluted in 18 μL of a 50% mixture of Qiagen buffer: ethanol.

Sample A was taken through the experimental method for channel based extraction using a 300 mm channel. A total of 7 elutions were collected. The procedure was repeated using sample B and processed in the same way, but on a device with a channel length of 1000 mm. Sample C was not processed through the channel but instead used as a positive control. The control sample and all elutions derived from each sample underwent PCR and gel separation.

The peak areas for each elution, for each sample were calculated and compared to the total peak area derived from the 2 ng control DNA sample, (170,000—total peak area). This data is shown in Table 8.

TABLE 8
Shoes the peak area and % recovery of each elution,
for each sample as compared to the control.

| | 300 mm channel | | 1000 mm channel | |
|---|---|---|---|---|
| | Peak Area | % Recovery | Peak Area | % Recovery |
| Elution 1 | 38162 | 43.09% | 77581 | 87.61% |
| Elution 2 | 1273 | 1.44% | 3367 | 3.80% |
| Elution 3 | 0 | — | 258 | 0.29% |
| Elution 4 | 0 | — | 0 | — |
| Elution 5 | 0 | — | 0 | — |
| Elution 6 | 0 | — | 0 | — |
| Elution 7 | 0 | — | 0 | — |
| TOTAL | 39435 | 44.53% | 81206 | 91.7% |

The peak areas for elutions 1-4 for each DNA sample are plotted in a bar graph as shown in FIG. 11. The 300 mm channel has successfully extracted and recovered approximately 45% of the total amount of DNA added. This value increases significantly to ≈92% recovery when using the longer 1000 mm channel. Clearly the channel length is a contributing factor to the amount of DNA that can be recovered from a sample and there is a risk that when samples contain large amounts of DNA, shorter channels become saturated and therefore cannot trap as much DNA as longer channels. However, this feature potentially offers a facility for addressing the problems which occur if DNA is too concentrated in the sample amplified. If a sample contains excess amounts of DNA, the resulting PCR will be over amplified thus making interpretation difficult.

Concentration measurements with a view to preventing this problem are difficult to achieve in DNA analysis. The use of a channel length, however, offers the possibility of placing an upper limit on the amount of DNA which is extracted and then eluted into a known eluent volume. Hence, control on the upper concentration limit is achieved. The results obtained in this example suggest that a channel has a fixed binding capacity and therefore any excess will not be retained. PCR can be optimised to amplify this maximum amount and therefore should not produce over amplified products.

### Example 8

### Illustrating the Saturation of a 30 cm Channel

In the previous example, it was seen that increasing the channel length increased the amount of DNA that could be recovered. This suggests that channels of a fixed dimension and therefore a predefined surface area, bind a specific amount of DNA. This implies that it may be possible to saturate a channel of fixed surafce area with DNA such that not further binding of DNA can occur.

To investigate this issue, a number of different DNA concentration within the range 0.1-0.5 ng/μL were prepared. In each case, 10 μL of sample was introduced and then taken through the experimental method for channel based extraction using a 300 mm channel. A total of 6 elutions were collected. Since SGM+PCR is optimised for only 2 ng total DNA, attempting to amplify more that 2 ng would not yield a quantitative linear relationship between peak area and total DNA amounts, therefore to address the issue of having too much template for PCR, for starting template concentration of 0.25, 0.3, 0.35 and 0.4, elution 1 was split into two aliquots prior to PCR. For starting DNA concentrations of 0.45 and 0.5 ng, elution 1 was split into three aliquots prior to PCR. Exactly the same treatment was given to control DNA samples which were not processed through the channel. Following PCR and gel separation, the peak areas for all sample were calculated. Where the sample was initially split prior to PCR, the resulting peak areas were summed, giving a total peak area for that specific DNA concentration.

The peak areas for each elution, for each sample are shown in Table 9

TABLE 9
shows the total Peak areas for DNA samples processed through the channel
(test) and total peak areas not processed through the channel (Control)

| | DNA Concentration ng/μL | | | | | | | | | |
|---|---|---|---|---|---|---|---|---|---|---|
| Total Peak Area | 0.1 | 0.15 | 0.175 | 0.20 | 0.25 | 0.3 | 0.35 | 0.40 | 0.45 | 0.50 |
| Test | 33752 | 62797 | 78618 | 123240 | 147213 | 261443 | 277625 | 267858 | 288099 | 280895 |
| Control | 41179 | 98327 | 119536 | 190136 | 392458 | 399294 | 540813 | 543739 | 672657 | 800911 |

The results from table 9 are plotted in FIG. 12. These results dearly show that the amount of expected PCR product as indicated by the total peak area for the control samples, continue to rise. There is a linear relationship. The results for the test sample however, increase up to approximately 0.3 ng/μL (3 ng total DNA). Above 3 ng total DNA, no increase in peak area is observed suggesting that the saturation limit for a 30 cm channel is 3 ng. This observation suggests that the channel could be used for two different aspects:

i) A small fixed channel length may provide a maximum optimum binding capacity to trap DNA (eg. 2 ng) from very concentrated samples. The amount of DNA which is recovered is known to be optimum for PCR and so reduces the possibility of having a compromised PCR result in subsequent analysis. In effect the channel is acting as a quantitative device.

ii) A long channel could be used to trap DNA from very dilute samples. Previous results have shown that longer channels trap more DNA. This is inferred by these results.

1-32. (canceled)

33. A method of controlling concentration of DNA extracted from a sample, the method including:
providing an extraction channel, the extraction channel having a known DNA retention capacity;
introducing the sample containing DNA to the extraction channel, the amount of DNA potentially exceeding the retention capacity of the extraction channel;
passing the sample through the extraction channel and removing the sample from the extraction channel, DNA being retained by the channel up to the retention capacity of the channel and thereby being extracted from the sample;
subjecting the extracted DNA to one or more further process steps to elute the extracted DNA into a post-extraction eluent, the post-extraction eluent being of known volume and thereby containing DNA of a known concentration;
using the eluent to provide an optimum amount of DNA to a subsequent method step.

34. The method according to claim 33 in which the sample is a whole blood sample.

35. The method according to claim 33 in which the subsequent method step is PCR.

36. A method of extracting DNA from a sample, the method including:
providing an extraction channel, the extraction channel having a DNA retention capacity;
introducing the sample containing DNA to the extraction channel, passing the sample through the extraction channel and removing the sample from the extraction channel, at least a part of the DNA being retained by the channel and thereby being extracted from the sample; and
subjecting the extracted DNA to one or more further process steps to elute the extracted DNA into a post-extraction eluent, the post-extraction eluent containing DNA, wherein the amount of DNA in the post-extraction eluent is less than or equal to the retention capacity of the extraction channel.

37. The method according to claim 36 in which the amount of DNA in the post-extraction eluent is equal to the retention capacity of the extraction channel.

38. The method according to claim 36 in which the retention capacity is set so as to provide a particular maximum concentration of DNA in the eluent.

39. The method according to claim 36 in which an excess of DNA, compared with the retention capacity of the extraction channel, passes through the extraction channel.

40. The method according to claim 36 in which the amount of DNA in the post-extraction eluent is controlled to a desired level or amount, irrespective of the starting level or amount in the sample.

41. The method according to claim 36 in which the retention capacity of the extraction channel is defined by its surface area.

42. The method according to claim 36 in which the time taken for a sample to pass through the extraction channel is used to control the level of DNA retained.

43. The method according to claim 36 in which the extraction channel consists only of the extraction channel walls.

44. The method according to claim 43 in which the extraction channel is free of beads or projections.

45. The method according to claim 36 in which the surface area of the extraction channel is pre-defined so as to extract a pre-defined amount of DNA from the sample.

46. The method according to claim 36 in which the surface area of the extraction channel is predetermined by adjusting the channel length.

47. The method according to claim 36 in which impurities are left in the sample and removed with the sample, whilst DNA from the sample is retained within the extraction channel and is not removed with the sample.

48. The method according to claim 47 in which the impurities include PCR inhibitors.

49. The method according to claim 47 in which the impurities left in the sample are dissolved species and/or suspended species and/or solid material.

50. The method according to claim 49 in which the impurities are one or more of: haem, lead incorporating materials, debris associated with cells.

51. The method according to claim 36 in which impurities remain in the sample as it passes through the extraction channel and the DNA is retained by the extraction channel.

52. The method according to claim 36 in which impurities bind irreversibly to silicon dioxide surfaces of the extraction channel as the sample passes through the extraction channel.

53. The method according to claim 36 in which the method includes one or more further steps, the one or more further steps including one or more of: cleaning, washing, PCR, cell destruction, analysis.
